(19) United States
(12) Patent Application Publication
(10) Pub. No.: US 2021/0393676 A1
(43) Pub. Date: Dec. 23, 2021

(54) APPLICATION OF HONOKIOL IN ANTI-OTOTOXICITY AND HEARING PROTECTION

(71) Applicant: Northwestern University, Evanston, IL (US)

(72) Inventors: Xiaodong Tan, Wilmette, IL (US); Claus-Peter Richter, Skokie, IL (US)

(73) Assignee: Northwestern University, Evanston, IL (US)

(21) Appl. No.: 16/639,669

(22) PCT Filed: Aug. 17, 2018
(86) PCT No.: PCT/US2018/046865

§ 371 Date: Feb. 17, 2020

Related U.S. Application Data
(60) Provisional application No. 62/546,846, filed on Aug. 17, 2017.

(51) Int. Cl. A61K 33/243; A61K 31/05; A61K 31/555; A61K 45/06; A61P 27/16

(52) U.S. Cl. CPC A61K 33/243; A61K 31/05; A61P 27/16; A61K 45/06; A61K 31/555

(57) ABSTRACT

Disclosed are methods and pharmaceutical compositions for treating and/or preventing hearing loss in a subject in need thereof. The disclosed methods typically utilize and the pharmaceutical compositions typical comprise an effective amount of a polyphenol therapeutic agent such as honokiol.

### CROSS-REFERENCE TO RELATED PATENT APPLICATIONS

The present application claims the benefit of priority under 35 U.S.C. § 119(e) to U.S. Provisional Application No. 62/546,846, filed on Aug. 17, 2017, the content of which is incorporated herein by reference in its entirety.

### BACKGROUND

The field of the invention relates to the use of anti-ototoxic therapeutics to treat and/or prevent induced and/or age-related hearing loss. In particular, the field of the invention relates to the use of polyphenol compounds such as honokiol for treating and/or preventing drug-induced, noise-induced, and/or age-related hearing loss.

Hearing loss is a growing problem with over 360 million patients worldwide suffering from this condition. Factors leading to an increase in hearing loss include an increasingly aging population, overexposure to noise in the youth and in the military, and exposure to ototoxic but lifesaving drugs such as aminoglycoside antibiotics and platinum-based chemotherapy. (See Peppi et al., Expert Op. Drug Delivery, (2018), 15:4, 319-324; the content of which is incorporated herein by reference in its entirety). The pathophysiology of hearing loss accordingly relates to the loss of hair cells in the inner ear, which are lost through aging, through exposure to loud noise, and through exposure to aminoglycoside antibiotics and platinum-based chemotherapy.

In order for sound to be perceived, sound first must enter the ear and the sound must be converted to a nerve impulse which is transmitted to the brain and is perceived as sound. (See website for American Speech-Language-Hearing Association). As such, sound travels to the brain first through sound waves which reach the outer ear and are conducted down the ear canal to the eardrum and cause the eardrum to vibrate. (See id.). Eardrum vibrations then are passed through tiny ear bones in the middle ear, which are the malleus, incus, and stapes and are collected referred to as the ossicles. (See id.). The ossicles then transfer the vibrations to fluid in the inner ear, which moves hair cells in the inner ear. (See id.). The movement of the hair cells converts the vibrations into nerve impulses, which are then transmitted to the brain by the auditory nerve. (See id.). The auditory nerve transmits the impulses to the brainstem, which subsequently transmits the impulses to the midbrain, which subsequently transmits the impulses to the temporal lobe to be interpreted as sound. (See id.).

As such, hair cells in the inner ear are a critical component for sound perception. Hair cells are gradually lost through aging and do not grow back on their own. Similarly, hair cells are lost through exposure to loud noises and through exposure to aminoglycoside antibiotics and platinum-based chemotherapy, and do not grow back on their own. Therefore, a therapy that can prevent the loss of hair cells that is experienced through aging, exposure to loud noises, or exposure to aminoglycoside antibiotics and platinum-based chemotherapy is desirable. Approximately one-third of the population over the age of sixty-five (65) will experience age-related hearing loss (ARHL). (See Peppi et al., Expert Op. Drug Delivery, (2018), 15:4, 319-324). For ARHL, there is no preventive treatment and curative treatments are limited to hearing aids and cochlear implants. (See id.)

It is estimated that over forty (40) million adults in the U.S. experience noise-induced hearing loss (NIHL). (See id.) For NIHL, preventive treatment is limited to avoiding risks of exposure to loud noises, and curative treatments are limited to treatment with corticosteroids, hearing aids and cochlear implants. (See id.)

It is estimated that approximately fifty percent (50%) of patients administered cisplatin will experience some hearing loss. Preventive treatment is limited to careful dosing of cisplatin, and there is no effective FDA-approved curative treatment.

Here, the inventors have shown that certain polyphenols, including honokiol, can be utilized to protect hearing against cisplatin-induce hearing loss. This is shown both in cultured cochlear primary cells and in animal studies. The inventors' findings have implications for therapeutics and methods for treating and/or preventing hearing loss.

### SUMMARY

Disclosed are methods and pharmaceutical compositions for treating and/or preventing hearing loss in a subject in need thereof. The disclosed methods typically utilize and the pharmaceutical compositions typical comprise an effective amount of a polyphenol therapeutic agent, such as honokiol.

The polyphenol therapeutic agent may be administered by any suitable method of administration, including but not limited to, oral administration, intraperitoneal administration, intravenous administration, and/or intracochlear and/or transtympanic drug delivery. Similarly, the pharmaceutical compositions comprising the effective amount of a polyphenol therapeutic agent may be formulated for delivery any suitable method of administration by any suitable method of administration, including but not limited to, oral administration, intraperitoneal administration, intravenous administration, and/or intracochlear drug and/or transtympanic delivery.

The disclosed methods may be practiced to prevent and/or treat hearing loss that is due to loss of hair cells of the cochlea. In some embodiments, the disclosed methods may be practiced to prevent and/or treat hearing loss that is due to induced loss of hair cells of the cochlea. Induced loss of hair cells may include noise-induced hearing loss (NIHL) that results in loss of hair cells of the cochlea.

Induced loss of hair cells also may include drug-induced hearing loss (DIHL) that results in loss of hair cells of the cochlea, which otherwise may be referred to as ototoxicity. In other embodiments, the disclosed methods may be practiced to prevent and/or treat ototoxicity that is induced by an antibiotic.

In other embodiments of the disclosed methods for preventing and/or treating ototoxicity, the ototoxicity may be induced by an administered platinum-based therapeutic. As such, in some embodiments of the disclosed methods for treating and/or preventing hearing loss in a subject in need thereof, the subject may have cancer and the subject may be administered and the subject also may be administered a polyphenol therapeutic agent, such as honokiol, or a pharmaceutical composition comprising the polyphenol therapeutic agent, either before, concurrently with, or after the subject is administered the platinum-based therapeutic.

### BRIEF DESCRIPTION OF THE DRAWINGS

FIG. 1. Illustrative mechanisms of cisplatin metabolism and toxicity borrowed from Langer et al., “Understanding platinum-induced ototoxicity,” Trends in Pharma. Sci., Volume 34, Issue 8, P458-469, Aug. 1, 2013).

FIG. 2. Illustration highlighting cochlear tissues that are prone to irreversible damage and cell death borrowed from Wong, A. C. & Ryan, A. F. Mechanisms of sensorineural cell damage, death and survival in the cochlea. Front Aging Neurosci 7, 58, doi:10.3389/fnagi.2015.00058 (2015). (See also Breglio et al., “Cisplatin is retained in the cochlea indefinitely following chemotherapy,” Nature Comm., 8 1654, 1-9, 2017).

FIG. 3. Chemical Structure of Honokiol.

FIG. 4 HNK activates Sirt3 and deacetylates mitochondrial proteins borrowed from Pillai et al., “Honokiol blocks and reverses cardiac hypertrophy in mice by activating mitochondrial Sirt3” Nat Commun 2015 Apr. 14; 6:6656). From Pillai et al., (a) Primary cultures of neonatal rat cardiomyocytes were treated with different doses of HNK as indicated. Mitochondrial lysate was prepared and analyzed for lysine-acetylation using an anti-acetyl lysine antibody (Ac-K). Total MnSOD level served as a loading control. (b) Primary cultures of neonatal rat cardiomyocytes were treated with 10 mM HNK at different time points as indicated. Mitochondrial lysate was prepared and analyzed for lysine-acetylation using an anti-acetyl lysine antibody. (c) Primary cultures of cardiomyocytes were treated with 5 and 10 mM HNK for 24 h. Cell lysate was analyzed by western blotting with indicated antibodies.

FIG. 5. SIRT3 regulates multiple intracellular pathways borrowed from Giralt and Villarroya, “SIRT3, a pivotal actor in mitochondrial functions: metabolism, cell death and aging,” Biochem. J. May 15, 2012. 444(1), 1-10. (See also Chen et al., “Sirtuin-3 (SIRT3), a therapeutic target with oncogenic and tumor-suppressive function in cancer,” Cell Death and Disease (2014) 5, e1047).

FIG. 6. SIRT3 role in Caloric Restriction Mediated Prevention of Hearing Loss borrowed from Someya et al., “Sirt3 Mediates Reduction of Oxidative Damage and Prevention of Age-related Hearing Loss under Caloric Restriction,” Cell. 2010 Nov. 24; 143(5): 802-812.

FIG. 7. SIRT3 in cancer cell. SIRT3 can limit ROS levels in cancer cell, thereby leading to the destabilization and subsequent degradation of HIF-1α borrowed from Chen et al., “Sirtuin-3 (SIRT3), a therapeutic target with oncogenic and tumor-suppressive function in cancer,” Cell Death and Disease (2014) 5, e1047.

FIG. 8. SIRT3 has as an oncogenic or a tumor-suppressive role in cancer borrowed from Chen et al., “Sirtuin-3 (SIRT3), a therapeutic target with oncogenic and tumor-suppressive function in cancer,” Cell Death and Disease (2014) 5, e1047.

FIG. 9. House Ear Institute-Organ of Corti 1 (HEI-OC1) cell survival after cisplatin and honokiol (HNK) treatment. (A) C: cisplatin; H: HNK. The number following the letter “C” and the letter “H” indicates the concentration in μM of cisplatin and honokiol, respectively. Cisplatin and honokiol were added simultaneously. The data were normalized to the number of cells in the negative control (COHO). (B) Western blot of protein indicated on left hand figure label after treatment with the indicated concentration of cisplatin, HNK or cisplatin+HNK.

FIG. 10. Effects of cisplatin and honokiol to the survival of different cancer cells. C: cisplatin; H: honokiol. The number following indicates the concentration in μM, (e.g., C100H0 means cisplatin 100 μM and honokiol 0 μM). Each treatment was repeated in triplets. The data were normalized by the number of untreated control (C0H0).

FIG. 11. Dose-response of cisplatin treatment (A to C) and effect of HNK pre-treatment (D to F) in terms of ABR TS in response to clicks and tones. *: P<0.05; **: P<0.01 compared to Day 0. Average=256. The error bars of all data points are standard deviations.

FIG. 12. HNK protection against cisplatin-induced changes of DPOAE amplitude. DPOAE decreased significantly at middle to high frequency ranges (over 16 kHz), example shown in (A). The DPOAE shift, normalized to Day 0, of all the groups are plotted in (B to E). The results of the 2 animals in Cis 15 mg/kg group on Day 14 were both plotted in (B) as the dashed lines and open circles. average=3000.

FIG. 13. Confocal images of the immunostaining (merged) of cochleae treated with Cis 15 (A) and Cis 15+HNK 20 (B) mg/kg, under 20× objectives.

FIG. 14. High-resolution confocal images (60× subjective) of cochleae treated with Cis 15 (Left) and Cis 15+HNK 20 (Right), taken at the locations marked by white squares in FIGS. 13A and B, respectively. The white arrows indicate the nuclei of the remaining OHCs and the hollow arrows indicate missing OHCs. Note the panels for hair bundles (phalloidin staining) might be off focus and are only for indicating the correct position of the bodies and nuclei of the OHCs.

FIG. 15. Weight loss of the animals treated with cisplatin 15 mg/kg with or without honokiol (Left) and daily animal survival rate of the animals treated with cisplatin with or without honokiol (Right). Significant weight loss was observed in cisplatin treated animals while largely improved by honokiol pre-treatment. The survival rate dropped down to zero in the cisplatin 20 mg/kg group at day 8, while also improved significantly with honokiol. Note that part of the animal loss was due to significant weight loss (over 25%) which were removed from the study following the animal protocol.

### DETAILED DESCRIPTION

The present invention is described herein using several definitions, as set forth below and throughout the application.

### Definitions

Unless otherwise specified or indicated by context, the terms “a”, “an”, and “the” mean “one or more.” For example, “a component” should be interpreted to mean “one or more components.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject,” “patient,” and “individual” may be used interchangeably and may refer to human and non-human animals. A subject in need thereof may include a subject having or at risk for experiencing hearing loss including hearing loss due to ototoxicity. A subject in need thereof may include a subject experiencing or at risk for experiencing ototoxicity caused by reactive oxygen species and resulting in apoptosis of hair cells of the cochlea and/or apoptosis of spiral ganglion neurons.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating hearing loss.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

“Ototoxicity” may include “induced ototoxicity,” including ototoxicity induced by administering anti-cancer agents such as platinum-containing anti-cancer agents to the subject in need thereof. Platinum-containing anti-cancer agents may include, but are not limited to, cisplatin, carboplatin, and oxaliplatin.

“Ototoxicity” may include induced ototoxicity, including ototoxicity induced by administering antibiotic agents to the subject in need thereof. Antibiotic agents may include but are not limited to aminoglycoside antibiotic agents such as aminoglycoside antibiotic agents that cause sensorineural hearing loss (SNHL). Aminoglycoside antibiotic agents may include, but are not limited to kanamycin, amikacin, tobramycin, dibekacin, gentamicin, sismicin, netilmicin, neomycin, and streptomycin.

A subject in need thereof may include a subject having or at risk for experiencing hearing loss due to noise exposure. Noise exposure may include occupational based noise exposure or other noise exposure

A subject in need thereof may include a subject having or at risk for developing age-related hearing loss. In some embodiments, the subject may be elderly, for example a subject having an age greater than 60, 65, 70, 75, 80, 85, 90, or older.

A subject in need thereof may include a subject having cancer or at risk for developing cancer. Cancers may include, but are not limited to, cancers that are treated by administering a platinum-containing chemotherapeutic agent. Cancers may include, but are not limited to, cancers characterized by solid tumors, including, but not limited to prostate cancer, breast cancer, colon cancer, head and neck squamous cell carcinoma, bladder cancer, epithelial cancer, hepatocellular carcinoma.

The disclosed methods may include methods of administering a polyphenol compound to a subject in need thereof. Polyphenol compounds may include bi-phenol compounds having a formula:

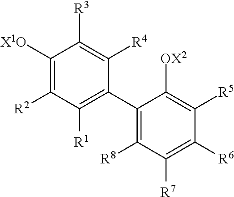

wherein X¹ and X² are H or C1-C6 alkyl, and R¹, R², R³, R⁴, R⁵, R⁶, R⁷, and R⁸ are selected from H, C1-C6 alkyl, C1-C6 alkenyl, C1-C6 alkoxy, and C1-C6 hydroxyalkyl. In particular, the bi-phenol compounds may include honokiol having a formula:

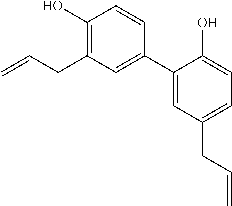 [SMILES: C=CCc1ccc(O)c(-c2ccc(O)c(CC=C)c2)c1]

and referred to as 2-(4-hydroxy-3-prop-2-enyl-phenyl)-4-prop-2-enyl-phenol.

In some embodiments, the disclosed subject matter relates to therapeutic uses of honokiol. Therapeutic uses for honokiol are disclosed in the art. (See, e.g., U.S. Publication Nos. 20090253634; and 20080300298; U.S. Pat. Nos. 8,822,531; 8,779,090; and 6,923,992; Hearing Loss: The Otolaryngologist's guide to Amplification (2010), Chapter 11. Nutritional Supplements for the Hearing-Impaired, Michael J. A. Robb and Michael D. Seidmann (Page 145); Cheng et al., “Synergistic antitumor effects of liposomal honokiol combined with cisplatin in colon cancer models” Oncol Lett. 2011 Sep. 1; 2(5): 957-962; Jiang et al., “Improved therapeutic effectiveness by combining liposomal honokiol with cisplatin in lung cancer model” BMC Cancer 2008, 8:242; Luo et al., “Liposomal honokiol, a promising agent for treatment of cisplatin-resistant human ovarian cancer” J Cancer Res Clin Oncol 2008 134(9):937-45; and Pillai et al., “Honokiol blocks and reverses cardiac hypertrophy in mice by activating mitochondrial Sirt3” Nat Commun 2015 Apr. 14; 6:6656, the contents of which are incorporated herein by reference in their entireties).

In some embodiments of the disclosed methods for treating and/or preventing hearing loss in a subject in need thereof, the subject may have cancer. The subject may be administered an effective amount of cisplatin for treating the cancer (e.g., a dose resulting in a concentration of as high as 10 μM, 20 μM, 30 μM, 40 μM, 50 μM, 60 μM, 70 μM, 80 μM, 90 μM, 100 μM, or higher in the subject, or a concentration range bounded by any of the foregoing values in the subject (e.g., 10-50 μM)). The subject also may be administered an effective amount of a polyphenol compound such as honokiol for treating and/or preventing ototoxicity of cisplatin (e.g., a dose resulting in a concentration as low as 50 μM, 45 μM, 40 μM, 35 μM, 30 μM, 25 μM, 20 μM, 15 μM, 10 μM, 5 μM, 1 μM or lower in the subject, or a concentration range bounded by any of the foregoing values in the subject (e.g., 20-5 μM)).

In some embodiments of the disclosed methods for treating and/or preventing hearing loss in a subject in need thereof, the subject may be a human having cancer. The subject may be administered an effective amount of cisplatin for treating the cancer (e.g., a dose as high as 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, or higher in the subject, or a dose range bounded by any of the foregoing values (e.g., 25-50 mg/kg)). The subject also may be administered an effective dose of honokiol for treating and/or preventing ototoxicity of cisplatin (e.g., a dose as low as 50 mg/kg, 45 mg/kg, 40 mg/kg, 35 mg/kg, 30 mg/kg, 25 mg/kg, 20 mg/kg, 15 mg/kg, 10 mg/kg, 5 mg/kg, 4 mg/kg, 3 mg/kg, 2 mg/kg, 1 mg/kg or lower in the subject, or a concentration range bounded by any of the foregoing values (e.g., 50-5 mg/kg)).

Honokiol and/or other therapeutic agents disclosed herein may be administered as pharmaceutical compositions and, therefore, pharmaceutical compositions incorporating the honokiol and/or other therapeutic agents are considered to be embodiments of the subject matter disclosed herein. Such pharmaceutical compositions may take any physical form which is pharmaceutically acceptable. Illustratively, pharmaceutical compositions can be systemically administered (e.g., via oral or parenteral administration such as intramuscularly (IM), subcutaneously (SC) and intravenously (IV)) or locally administered (e.g., via intracochlear injection, transtympanic injection, and/or an intra-cochlear drug delivery device). (See Peppi et al., “Intracochlear drug delivery systems: a novel approach whose time has come,” Expert Op. Drug Delivery, (2018), 15:4, 319-324; and Liu et al., “Current strategies for drug delivery to the inner ear,” Acta Pharmaceutica Sinica B 2013; 3(2):86-69; the contents of which are incorporated herein by reference in their entireties). Deliver methods may include, but are not limited to inter-tympanic (IT) delivery of solutions or controlled release matrices to the Round Window Membrane (RWM), osmotic pumps (see Brown J N, Miller J M, Altschuler R A, et al., “Osmotic pump implant for chronic infusion of drugs into the inner ear,” Hear Res. 1993; 70(2):167-172), magnetic nanoparticles, cochlear prosthesis-mediated delivery (see Staecker H, Jolly C, Garnham C. “Cochlear implantation: an opportunity for drug development,” Drug Discov Today. 2010; 15(7-8):314-321), microneedle-based penetration of the RWM (see Watanabe H, Cardoso L, Lalwani A K, et al., “A dual wedge microneedle for sampling of perilymph solution via round window membrane,” Biomed Microdevices. 2016; 18(2): 24. PMCID: 5574191), and constant infusion intracochlear delivery systems (see Borkholder D A, Zhu X, Hyatt B T, et al., “Murine intracochlear drug delivery: reducing concentration gradients within the cochlea,” Hear Res. 2010; 268(1-2): 2-11. PMCID: 2933796

Such pharmaceutical compositions contain an effective amount of honokiol and/or other therapeutic agents, which effective amount may be related to the daily dose of the compound to be administered. Each dosage unit may contain the daily dose of a given compound or each dosage unit may contain a fraction of the daily dose, such as one-half or one-third of the dose. The amount of honokiol and/or other therapeutic agents to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of honokiol and/or other therapeutic agents after administration to the patient by employing well known procedures.

A typical daily dose may contain from about 0.01 mg/kg to about 100 mg/kg (such as from about 0.05 mg/kg to about 50 mg/kg and/or from about 0.1 mg/kg to about 25 mg/kg) of honokiol and/or other therapeutic agents in the present methods of treatment. Compositions can be formulated in a unit dosage form, each dosage containing from about 1 to about 500 mg of honokiol and/or other therapeutic agents individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, and buccal routes. Other illustrative routes include intracochlear and transtympanic routes. The route of administration may be varied in any way, limited by the physical properties of the compounds being employed and the convenience of the subject and the caregiver.

As one skilled in the art will appreciate, suitable formulations include those that are suitable for more than one route of administration. Alternatively, suitable formulations include those that are suitable for only one route of administration as well as those that are suitable for one or more routes of administration, but not suitable for one or more other routes of administration.

### EXAMPLES

The following Examples are illustrative and are not intended to limit the scope of the claimed subject matter.

### Example 1—Honokiol for Treating and/or Preventing Ototoxicity

Abstract

Ototoxicity can be induced by multiple factors such as noise¹, antibiotics², aging³ and cisplatin⁴. Accumulative oxidative damage caused by reactive oxygen species (ROS) is a universal mechanism, which leads to apoptosis of hair cells (HC) and spiral ganglion neurons (SGN)^{5,6}. The loss of HCs is irreversible. Therefore, anti-ototoxic medicine is under intensive investigation⁷⁻¹⁰. Although some are under clinical trials, no ideal protective agent against ototoxicity has been widely used yet. Compromising the antitumor effect of cisplatin is the major concern^{7,10}. Toxicity by itself¹¹ and blood-inner ear barrier¹²⁻¹⁴ are the other two most common problems encountered (for review, see)¹⁵. In this patent application we claim a therapy for hearing protection against the insults using honokiol (HNK). HNK, a small molecule polyphenol, is a multifunctional antitumor and antiangiogenic agent¹⁶. HNK directly activates SIRT3¹⁷, a deacetylase exclusively expressed in mitochondria¹⁸, which in turn reduces ROS synthesis¹⁷. Evidence has shown that activation of SIRT3 reduced the oxidative damage also in cochlear hair cells in age-related hearing loss^{3,19}. Although the mechanism is not fully understood yet, we have observed significant protection by HNK against cisplatin-induced cell death in HEI-OC1 cells. HEI-OC1 is a cochlear-derived cell line most widely used in the screening of anti-ototoxicity medicine^{20,21}. Here, we disclose the anti-ototoxic protection effect of HNK, including but not limited to co-application of HNK in cisplatin chemotherapy and in cochlear implantation.

Applications

The applications of the disclosed technology may include, but are not limited to: (a) co-application with cisplatin in chemotherapy to reduce its ototoxic side effect; (b) application in cochlear implant surgery for protection of cochlear cells, such as neurons and remaining hair cells; (c) application before, during and after drug or noise exposure for hearing protection; (d) routine uptake for protection against age-related hearing loss.

Advantages

The applications of the disclosed technology may include, but are not limited to: (a) high efficiency: almost 100% protection against high dose cisplatin ototoxicity (50 μM) at low concentration (10 μM). HNK meets all other requirements of an ideal anti-ototoxicity medicine such as membrane permeability, small molecular weight, non-toxic and no interference with cisplatin in terms of molecular mechanism. In addition, HNK can also cross the blood brain barrier and blood cerebrospinal fluid barrier (BCSFB), an alternative way for drug delivery. Finally, HNK is already in pre-clinical trials for its anti-tumor activity.

### BRIEF SUMMARY

The disclosed subject matter relates to the therapeutic effect of HNK on hearing protection. In particular, HNK is disclosed for preventing cochlear cell death triggered by cisplatin application. Cisplatin is a widely used chemotherapeutic agent with dose limiting side effects including ototoxicity^{4,15,22,23}. HEI-OC1, a cochlea derived cell line widely used for drug screening for hearing protection or hearing loss treatment, is sensitive to cisplatin treatment in a dose-dependent manner^{20,24,25}.

Others have attempted to address the ototoxicity of cisplatin by administering anti-ototoxic agents and have experienced various problems, including: (a) interference of the anti-ototoxic agents with the antitumor effects of cisplatin^{7,10,26}; (b) cell toxicity¹¹; (c) and efficiency of the anti-ototoxicity agent (i.e., a low therapeutic index of the ototoxic agent because of the failure to pass through the blood-inner ear barrier¹²). We have found that the use of HNK as an anti-ototoxic agent addresses all of these problems. HNK does not interfere with the antitumor effect of cisplatin. In fact, HNK itself is undergoing pre-clinical trial for its antitumor activity. HNK is cell membrane¹⁷ and blood-brain barrier^{27,28} permeable and has a smaller molecular weight (266 kD) compared to other candidate compounds for anti-ototoxic therapeutics. Finally, our pilot study shows that no cell toxicity is found at the concentration of highest effect for honokiol (10 μm) in protecting cells against cisplatin.

As such, HNK has the potential to be the first widely used anti-ototoxic therapeutic that is effective against multiple insult factors. In particular, the co-application of HNK in cisplatin chemotherapy has critical clinical significance. HNK has the potential to become the standard treatment for hearing protection in cancer therapy. In addition, HNK may be administered to protect remaining HCs during cochlear implant surgery during which HCs experience oxidative stress. Finally, administration of HNK immediately after hearing insult may be used to protect the loss of HCs.

### Example 2—Understanding the Mechanism of Cochlear Cell Protection by Honokiol (HNK) in Cisplatin Treatment

Specific Aims

Platinum drugs are extremely powerful and effective for solid cancers and are used in 40% of chemotherapy. However, the adverse side-effects of platinum drugs limit the dose at which they may be administered to achieve maximum anti-cancer benefit^{4,15,22,23}. The first and most widely used platinum drug, cisplatin, can cause nausea, vomiting, kidney failure, and hearing and balance related problems including significant hearing loss^{4,29}, tinnitus and vertigo. Ototoxicity of cisplatin is thought to be related to the generation of reactive oxygen species (ROS), which accumulate in hair cells (HC) due to compromised mitochondria function and lead to apoptosis¹⁻⁵. Intensive investigation on anti-ototoxic therapeutic to reduce this adverse side effect of platinum drugs is underway, although no ideal protective agent has been widely used yet. Toxicity of the anti-ototoxic therapeutics themself¹¹, interference with the antitumor effect of cisplatin^{7,10} and the inability of some anti-ototoxic therapeutics to pass the blood-inner ear barrier are the most common problems encountered.¹⁵ Although local delivery through transtympanic injection may address some of these problems¹², adverse effects in other tissue or organs still are encountered, such as neurotoxicity and nephrotoxicity.

Here, we propose the use of honokiol (HNK) for use in protecting cochlear hair cells against cisplatin ototoxicity. HNK, a small-molecule polyphenol, is a multifunctional antitumor, anti-inflammatory and antiangiogenic agent¹⁶ which can pass through the blood-brain barrier¹⁷. Recently, it was shown in cardiac fibroblast cells that HNK functions through direct activation of sirtuin-3 (SIRT3), the primary NAD⁺-dependent deacetylase in mitochondria¹⁷. In normal cells, SIRT3 can decrease the production of ROS through the deacetylation and activation of manganese-dependent superoxide dismutase (MnSOD). It also has been shown in cochlea hair cells that the activation of SIRT3 can reduce the oxidative damage in age-related hearing loss³. In cancer cells, however, the SIRT3-MnSOD-ROS axis is dysregulated and the oxidative stress is high, which are in association with their metabolic reprogramming, transformation and resistance development. The activation of SIRT3 can reverse these processes, and therefore inhibit cancer cell proliferation. In our pilot studies, we observed significant protection of the House Ear Institute-Organ of Corti 1 (HEI-OC1) cells against cisplatin treatment by HNK. HEI-OC1 is a cochlear-derived cell line most widely used in the screening for anti-ototoxic therapeutics²⁰. The protective effect of HNK, however, was not shown in prostate cancer cells. Therefore, HNK has the potential to be co-applied with cisplatin in cancer treatment to protect hearing loss without compromising its therapeutic effects. Here, we propose further studies to confirm the oto-protection effect of HNK, and to reveal its sub-cellular mechanism. In addition, we propose further studies to determine whether the potential protection of HNK extends to other normal cells such as kidney cells. We hypothesize that, HNK can protect HEI-OC1 and human embryonic kidney (HEK) cells, but not cancer cells, from cisplatin induced-apoptosis through the activation of SIRT3. Three specific aims are designed for the study.

Aim I. To Confirm that HNK can Protect HEI-OC1 and HEK Cells from Cisplatin-Induced Apoptosis.

In our preliminary study, 10 μM HNK was able to completely prevent HEI-OC1 apoptosis induced by 50 μM cisplatin. Since HEI-OC1 cells express both hair cell and supporting cell marks as well as neural markers in our study, and the data was obtained only at 24 hours, we will confirm this result with cell viability, apoptosis and clonogenic assays using western blot. Immuno-fluorescence histology (IFH) will also be applied to label the hair cell marks (prestin, myosin 7a, Atoh1, etc.) and supporting cell marks (connexin 26, FGF-R, etc.). We would like to show, first, whether apoptosis is induced in prolonged culturing with cisplatin and HNK treatment, and second, whether HNK exhibits cell preference in regard to protection. Dose dependent curves of HNK protection will also be obtained. Meanwhile, similar treatment with cisplatin and HNK as in our preliminary study will also be performed in HEK293 cells. The protection of HEK293 cells against cisplatin-induced apoptosis by HNK will be determined through cell survival analysis. The results will provide more convincing evidence of the protective effect of HNK against cisplatin-induced cell death,

Aim II. To Confirm that HNK Exhibits Little or No Protection on Cancer Cells Against Cisplatin-Induced Apoptosis.

Our preliminary data also showed that HNK (up to 25 μM) did not prevent prostate cancer cell (C4-2B) death from 50 μM cisplatin treatment. In this proposal, we would like to provide further evidence to confirm that HNK exhibits little or no protection on cancer cells in cisplatin treatment. First, the dose response of the cancer cells to cisplatin co-treated with 10 μM HNK (or optimized dosage in HEI-OC1 and HEK cells) will be acquired. This study will provide more detailed information as to whether HNK protection of cancer cells is observed at lower cisplatin concentration. Second, the same study will be repeated in another ovary cancer cell line. This study will confirm that the lack of interference of HNK to cisplatin's antitumor effect is a general phenomenon.

Aim III. To Confirm the Protection Effect of HNK on HEI-OC1 Cells Against Cisplatin Insult Works Through Mitochondrial SIRT3.

It has been shown that HNK works through the activation of SIRT3 in cardiac fibroblasts. In this proposal we would like to determine whether the same molecular mechanism is involved in cochlear cells. In our pilot studies, we showed that SIRT3 expression level was elevated 24 hours after cisplatin treatment when HNK was administered. In addition, DNA damage was diminished in the presence of HNK. Although the expression of an early apoptosis mark, caspase-3, was higher in some cells co-treated with HNK and cisplatin versus cells treated with cisplatin alone, cell counting results suggested that these cells co-treated with HNK and cisplatin ultimately survived. Nevertheless, these preliminary results will be confirmed in the proposed studies. The expression and function of SIRT3 (and Sirt2 as well, as a control) in HEI-OC1 cells will be further confirmed using a series of cisplatin doses and HNK doses. Function of SIRT3 will be assessed, for example, by measuring the acetylation level of its substrates such as MnSOD and/or isocitrate dehydrogenase 2 (IDH2). The changes in the acetylation levels of SIRT3's substrates and HNK protection will also be investigated by the construction and transfection of anti-SIRT3 siRNA to the HEI-C1 cells.

The results will lay the ground work for the systematic co-application of HNK with cisplatin in chemotherapy, and will provide insight into more widely application of HNK in preventing hair cell loss such as in noise/drug-induced and age-related hearing loss.

### Example 3—Protective Effect of Honokiol Against Cisplatin Ototoxicity

Introduction

Cisplatin chemotherapy is used for treating solid tumors in >40% of cases. Adverse effects of cisplatin chemotherapy include nausea, vomiting, kidney failure, and hearing and balance related problems, such as significant hearing loss, tinnitus, and vertigo, which limit the usage and dosage of cisplatin that can be administered.

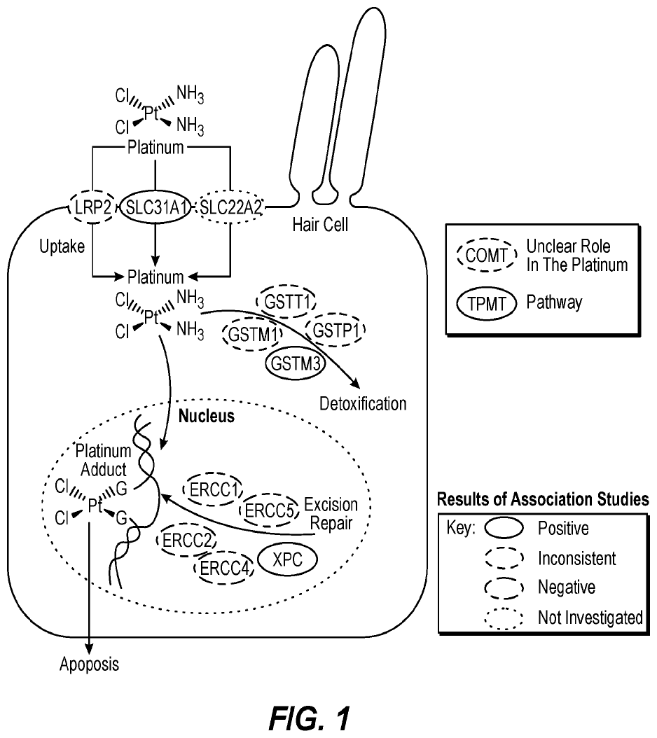

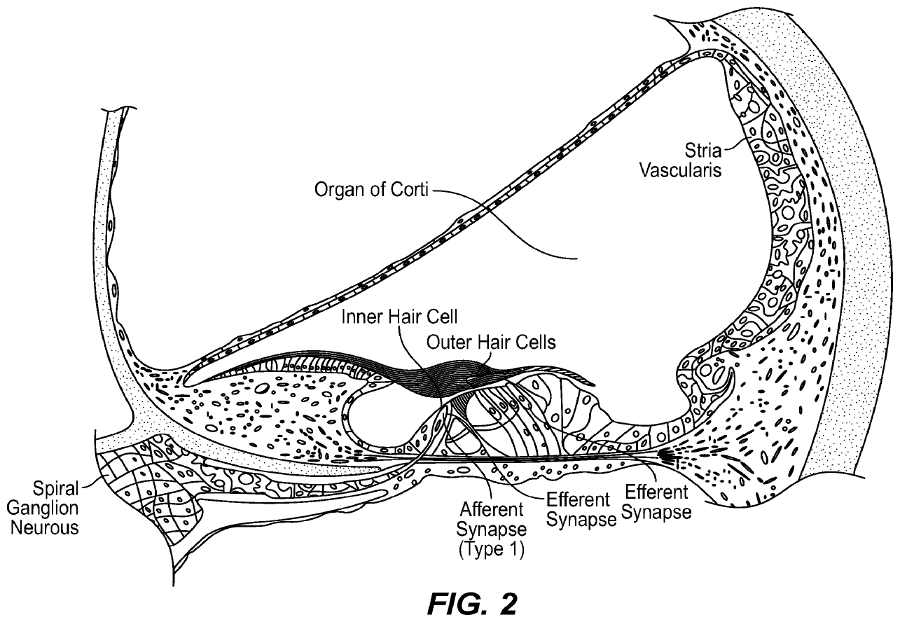

The mechanisms of action of cisplatin's antitumor toxicity include DNA cross-linking and generation of reactive oxygen species (ROS) which damage DNA and other biological molecules. (See FIG. 1 borrowed from Langer et al., “Understanding platinum-induced ototoxicity,” Trends in Pharma. Sci., Volume 34, Issue 8, P458-469, Aug. 1, 2013; the content of which is incorporated herein by reference in its entirety). Ototoxicity results from the cumulative oxidative damage caused by ROS, where progressive deterioration is observed primarily in the outer hair cells of the organ of corti, but also in the inner hair cells and supporting cells, spiral ganglion neurons (SGNs), the auditory nerve, stria vascularis and the spiral ligament. (See FIG. 2 borrowed from Wong, A. C. & Ryan, A. F. Mechanisms of sensorineural cell damage, death and survival in the cochlea. Front Aging Neurosci 7, 58, doi:10.3389/fnagi.2015.00058 (2015)). Because the cumulative oxidative damage is caused by ROS, candidate anti-ototoxic therapeutics include antioxidants. However, no ideal antioxidant treatment has yet been developed into a clinical treatment, because antioxidants can comprise the antitumor effect of cisplatin. Also, many antioxidants exhibit poor bio-availability and have limited efficacy in vivo, or the antioxidants may exhibit non-tumor toxicity themselves.

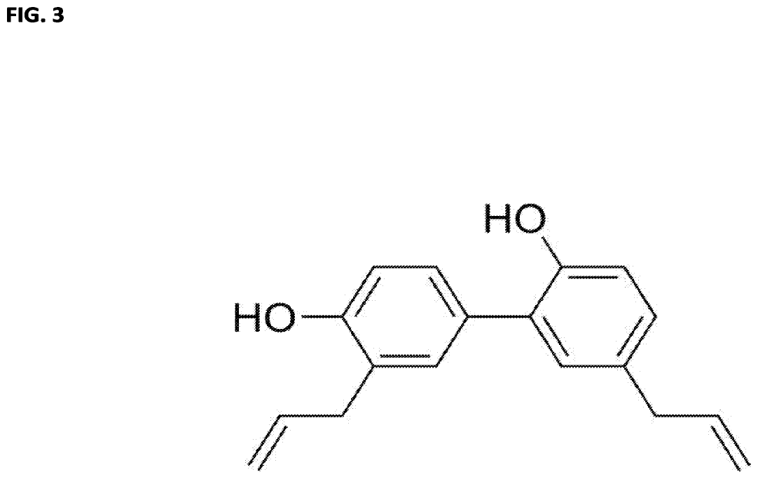

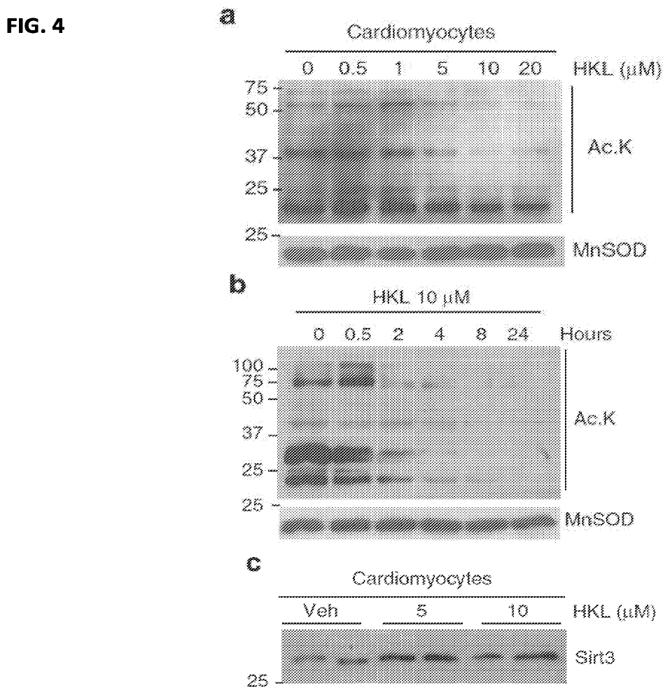

Honokiol is a bi-phenol compound present in magnolia bark. (See FIG. 3). Honokiol has been used in traditional Chinese medicine and has been observed to exhibit biological activities that include anti-angiogenic activity and anti-tumor activity. Honokiol has been observed to exhibit anti-cancer activity against cancers that include leukemia, colon cancer, lung cancer, and melanoma. Honokiol has been observed to exhibit synergy with cisplatin in cancer treatment, which synergy has been studied intensely without knowing the exact mechanism. Honokiol has been observed to protect normal tissues from oxidative damage including heart, liver, brain, and kidney by a mechanism of action that includes enhancing expression of the mitochondrial protein NAD-dependent deacetylase sirtuin-3 (SIRT3). (See FIG. 4 borrowed from Pillai, V. B. et al. Honokiol blocks and reverses cardiac hypertrophy in mice by activating mitochondrial Sirt3. Nat Commun 6, 6656, doi:10.1038/ncomms7656 (2015)). Honokiol is non-toxic and is capable of passing the blood-brain barrier.

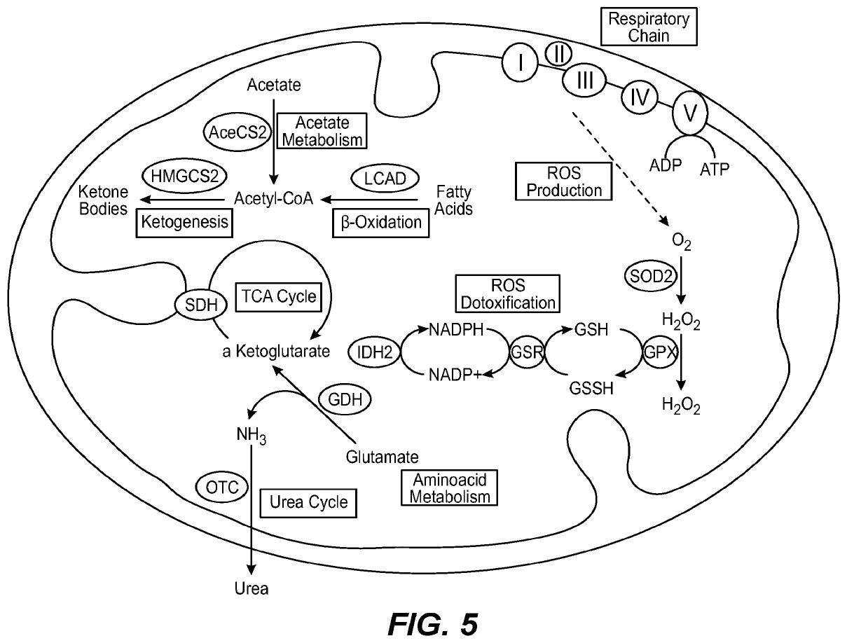

SIRT3 regulates multiple intracellular pathways. (See FIG. 5 borrowed from Giralt and Villarroya, “SIRT3, a pivotal actor in mitochondrial functions: metabolism, cell death and aging,” Biochem. J. May 15, 2012. 444(1), 1-10. (See also Chen et al., “Sirtuin-3 (SIRT3), a therapeutic target with oncogenic and tumor-suppressive function in cancer,” Cell Death and Disease (2014) 5, e1047). SIRT3 is involved in intracellular metabolic processes including energy production, ROS production and detoxification. SIRT3 is critical for maintaining mitochondrial integrity and function.

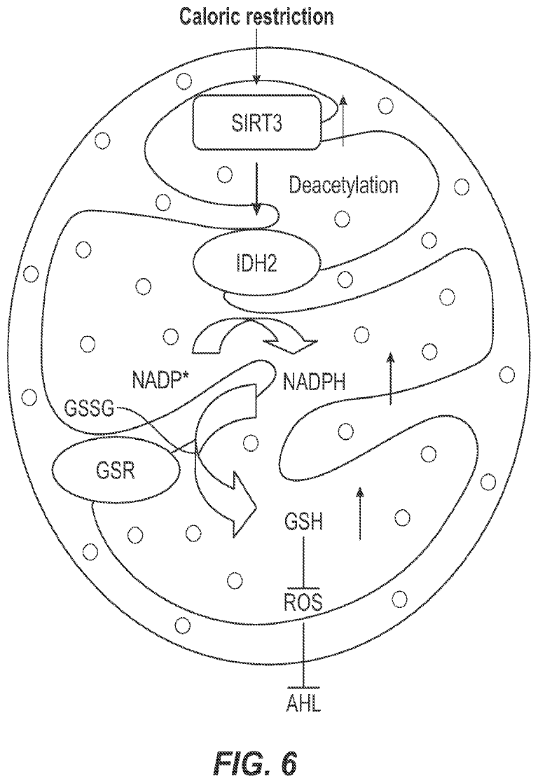

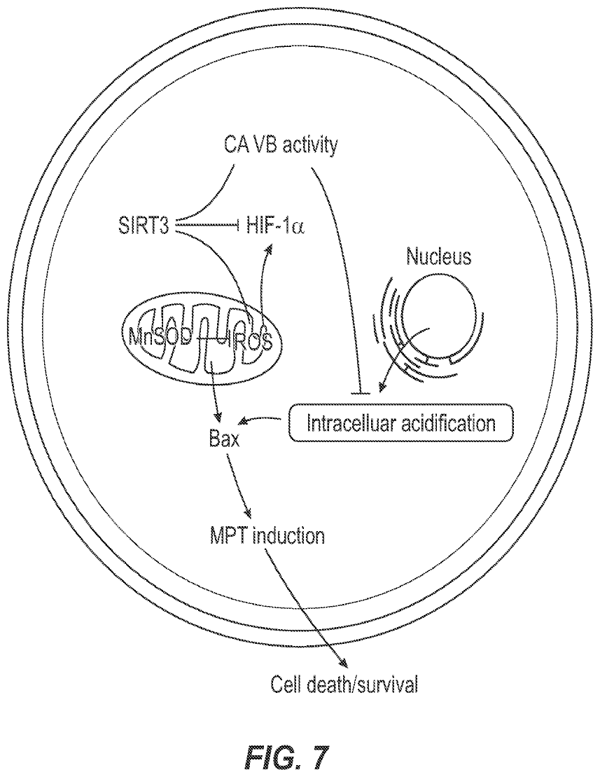

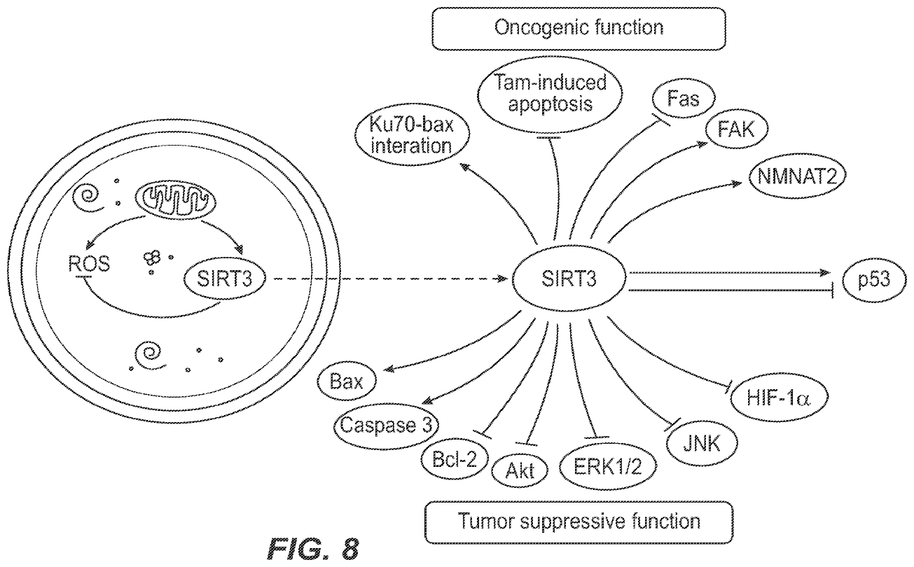

As such, SIRT3 exhibits roles in both of normal cells and cancer cells. (See Chen et al., “Sirtuin-3 (SIRT3), a therapeutic target with oncogenic and tumor-suppressive function in cancer,” Cell Death and Disease (2014) 5, e1047). In normal cells, SIRT3 prevent cell death from oxidative stress. In particular, SIRT3 is involved in hearing protection and prevention of age-related hearing loss under caloric restriction, through the deacetylation and activation of the mitochondrial protein isocitrate dehydrogenase (IDH2) and enhancement of the glutathione (GSH) antioxidant defense system. (See FIG. 6 borrowed from Someya, S. et al., “Sirt3 mediates reduction of oxidative damage and prevention of age-related hearing loss under caloric restriction,” Cell 143, 802-812, doi:10.1016/j.cell.2010.10.002 (2010)). In cancer cells, SIRT3 limits levels of ROS and leads to the degradation of hypoxia-inducible factor 1-alpha (HIF-1a). (See FIG. 7 and FIG. 8 borrowed from Chen et al., “Sirtuin-3 (SIRT3), a therapeutic target with oncogenic and tumor-suppressive function in cancer,” Cell Death and Disease (2014) 5, e1047).

Therefore, SIRT3 expression has contrary roles in the prevention of ototoxicity versus cisplatin-based chemotherapy as an oncogene and a tumor suppressor, and SIRT3 controls the balance between health and disease. SIRT3 exhibits oncogenic activity in rescuing p53-induced growth arrest and mediating resistance and carcinogenesis in cancer including head and neck squamous cell carcinoma, breast cancer, and bladder cancer among others. SIRT3 exhibits tumor suppressive activities that include pro-apoptotic activity in cancers that include human epithelial cancer, leukemia, and hepatocellular carcinoma among others.

In summary, cisplatin is widely used in solid tumor chemotherapy with significant adverse effects that limit usage and dosage, one of which being ototoxicity. Candidate drugs for treating or preventing ototoxicity include antioxidants. However, antioxidants present multiple problems and none are widely accepted in clinical practice.

Honokiol (HNK) potentially has great clinical value regarding hearing protection in chemotherapy. Honokiol may exhibit protection against ototoxicity and in addition honokiol is tumor suppressive in synergy with cisplatin. Instead of functioning as an antioxidant and free radical scavenger of ROS, HNK functions by increasing expression of mitochondrial SIRT3, which is an important target for cancer treatment and a novel target for hearing protection. Treatment with honokiol to prevent cisplatin-induced hearing loss may become a breakthrough in hearing protection and may potentially lead to a paradigm shift in cancer therapy.

Because honokiol improves mitochondrial function through SIRT3 to protect normal tissue and because outer hair cells of the organ of corti (OHC) are abundant in mitochondrial and sensitive to cisplatin, we propose that honokiol can prevent cisplatin-induced loss of OHC through increasing expression of SIRT3.

Study and Results

The following studies were performed to determine the otoprotective effect of honokiol against cisplatin ototoxicity in vivo, particularly in primary cochlear cells, without a corresponding reduction in efficacy of the anti-tumor effect of cisplatin against cancer cells. The following studies also were performed to determine the effect of honokiol treatment on cisplatin-induced auditory brainstem response (ABR) threshold shift and distortion product otoacoustic emission (DPOAE) amplitude decrease. The protective effect of honokiol against cisplatin ototoxicity was also studied by immunostaining and confocal microscopy to determine whether honokiol can prevent the reduction of outer hair cells (OHC) observed in cisplatin ototoxicity.

Protection of Primary Cochlear Cells

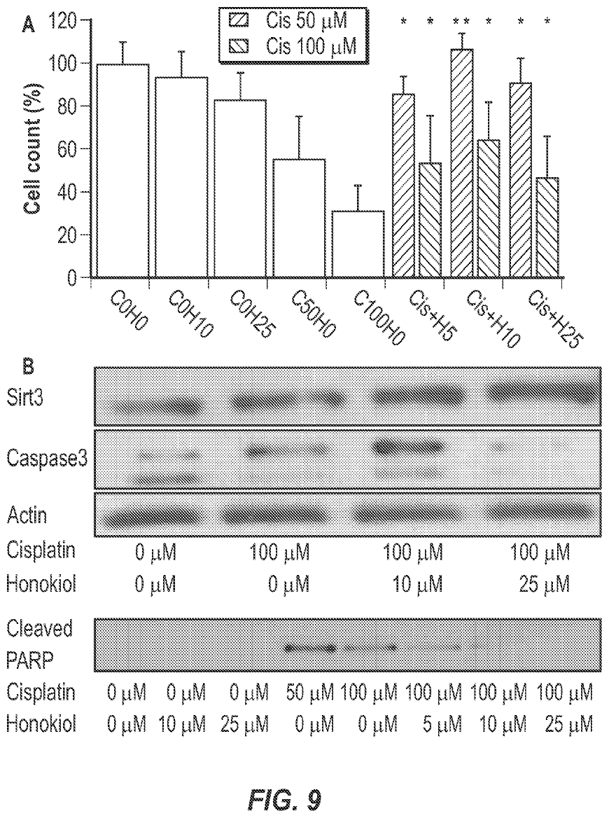

We tested the ototoxic effect of honokiol on House Ear Institute-Organ of Corti1 (HEI-OC1) cells, which are derived from a long-term culture of immortal mouse cochleae. Cells were treated with various concentrations of cisplatin (0 μm (C0), 50 μM (C50) or 100 μM (C100)) in the presence of various concentrations of honokiol (0 μm H0), 5 μM (H5), 10 μM (H10), or 25 μM (H25)). (See FIG. 9). Honokiol at a concentration of 5 μM or 10 μM was observed to protect HEI-OC1 cells against cisplatin-induced cell loss when cisplatin was administered at concentrations of 50 μM or 100 μM. (See FIG. 9). Only a minor decrease in cell count was observed when cells were treated with up to 10 μM honokiol alone. (See FIG. 9). As such, honokiol was observed to protect HEI-OC1 cells against cisplatin-induced cell loss. Honokiol also was observed to increase expression of SIRT3 in HEI-OC1 at increasing concentrations, and honokiol was observed to decrease expression of Caspase 3 and cleavage of poly ADP ribose polymerase (PARP), which are associated with apoptosis.

Honokiol Exhibits No Protective Effect Against Cisplatin on Three Different Cancer Cells Lines

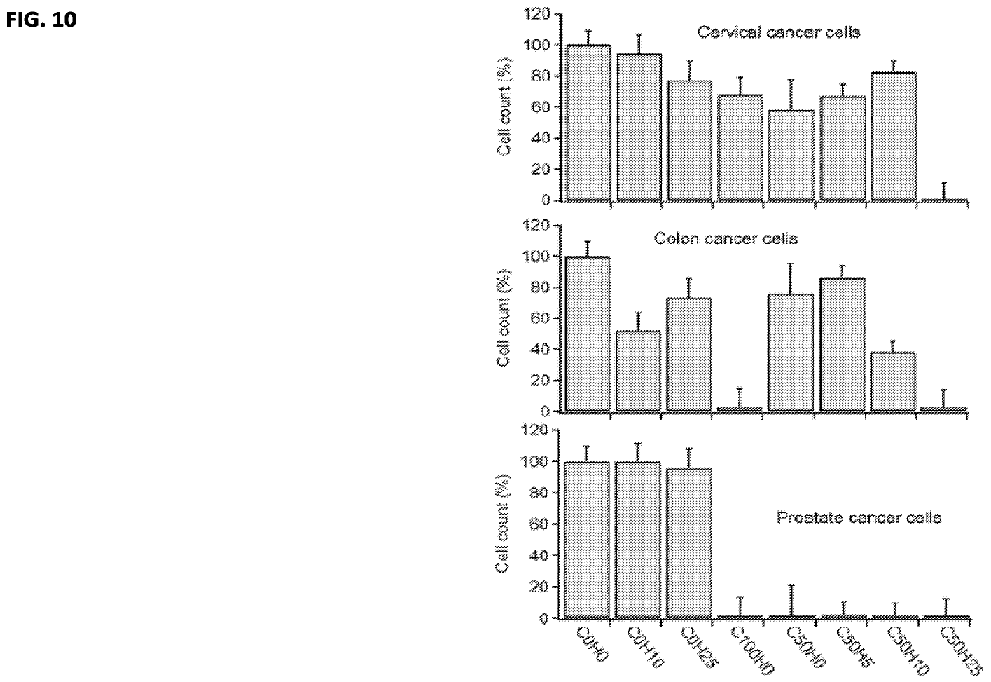

Because the role of SIRT3 may be different in different cancers, we selected three (3) different cancer cells for testing: prostate cancer cells, cervical cancer cells, and colon cancer cells. (See FIG. 10). Cisplatin was administered to the cancer cell lines with honokiol. Cisplatin induced a massive loss of prostate cancer cells at 50 μM (C50) or 100 μM (C100) and a massive loss of colon cancer cells at 100 μM (C100). Cisplatin only induced a mild reduction in cervical cancer cells at 50 μM (C50) or 100 μM (C100).

Honokiol also was co-administered with cisplatin to the cancer cell lines. Honokiol was not observed to protect any of the cancer cells from the anti-tumor toxicity of cisplatin. (See FIG. 10). Honokiol was observed to have a synergistic effect with cisplatin when honokiol was administered at 25 μM and cisplatin was administered at 50 μM (C50H25) to cervical cancer cells. (See FIG. 10). Honokiol also was observed to have a synergistic effect with cisplatin when honokiol was administered at 10 μM or 25 μM and cisplatin was administered at 50 μM (C50H10 and C50H10, respectively) to colon cancer cells. (See FIG. 10). Therefore, honokiol did not protect any of the cancer cell lines from the toxicity of cisplatin and honokiol and cisplatin exhibited synergy in killing cervical cancer cells and colon cancer cells.

Protection of Hearing Threshold: Cisplatin Induced a Dose-Dependent Auditory Brainstem Response (ABR) Threshold Shift (TS) that Diminished with Honokiol Pre-Treatment

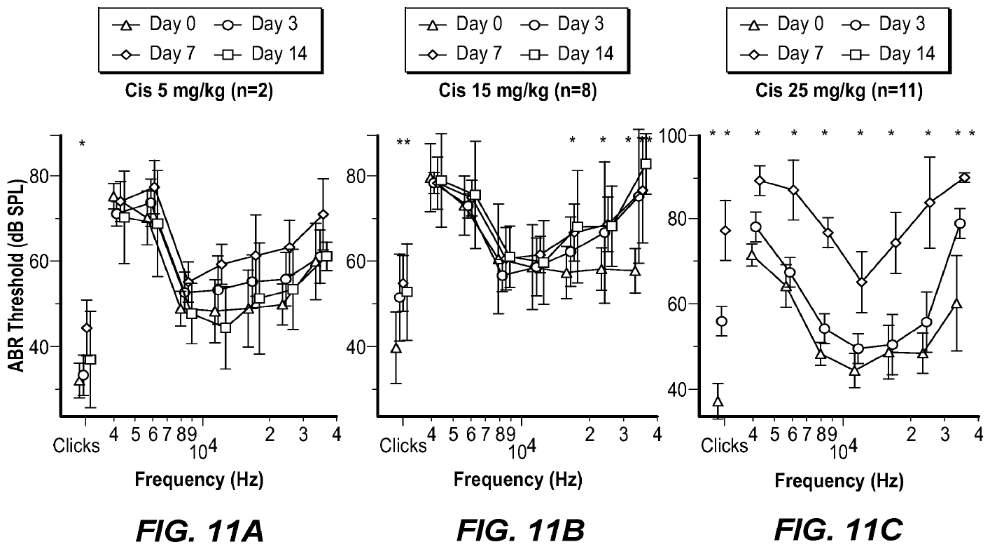

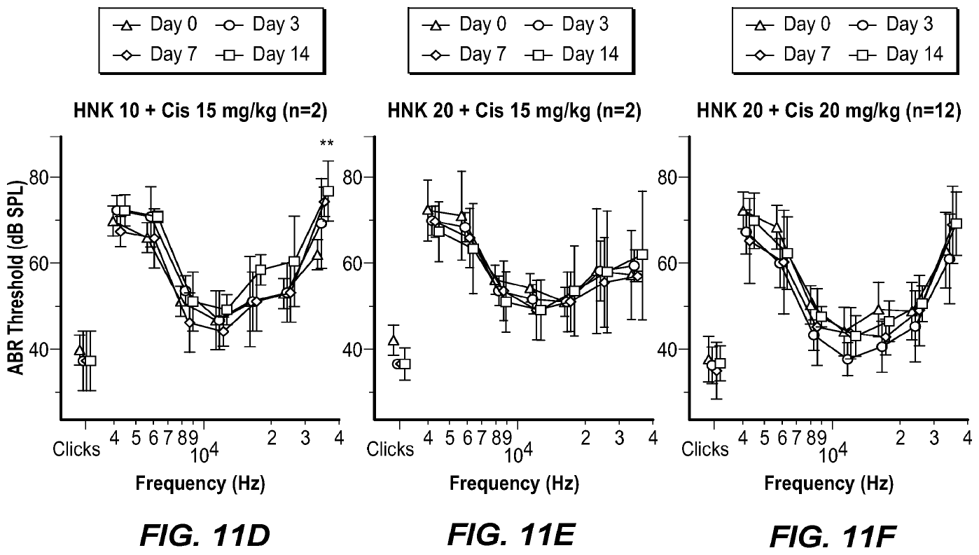

A dose-dependent Auditory Brainstem Response (ABR) Threshold Shift (TS) was induced in cisplatin-treated mice. (See FIG. 11). When cisplatin was administered to the mice at a dose of 5 mg/kg, a 5-15 dB TS was observed up to Day 7 and recovered at Day 14, in both click and tonal ABRs. However, higher doses of cisplatin (15 mg/kg and 20 mg/kg) induced more severe ABR TS in clicks and tones, which was not recovered after 14 days. Interestingly, when 15 mg/kg cisplatin was administered to the mice, TS was observed mainly at frequencies over 10 kHz, while systemic elevation of ABR threshold at all frequencies was shown when 20 mg/kg cisplatin was administered. In the mice pre-treated with honokiol (HNK), ABR TS induced by cisplatin decreased significantly. (See FIG. 11). Only the mice that were administered HNK 10 mg/kg and cisplatin 15 mg/kg showed a significant TS in tonal ABR at the highest frequency (32 kHz). No further significant TS was observed when different dosages of HNK (10-20 mg/kg) and cisplatin (15-20 mg/kg) were administered. In summary, HNK almost completely protects hearing threshold shift induced by cisplatin

HNK Prevents Cisplatin-Induced Distortion Product Oto-Acoustic Emission (DPOAE) Amplitude Decrease, Suggestion Outer Hair Cell (OHC) Protection

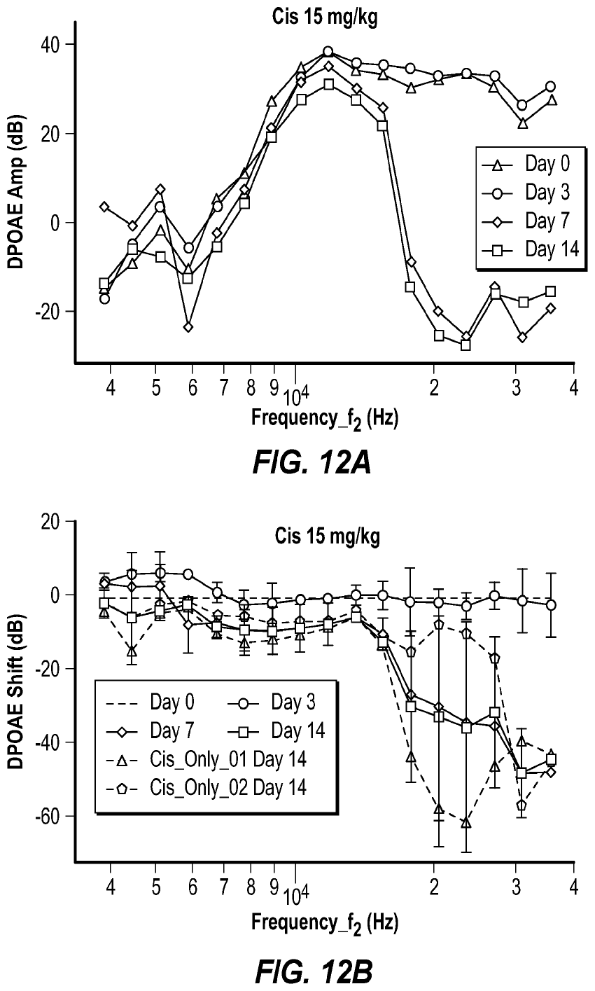

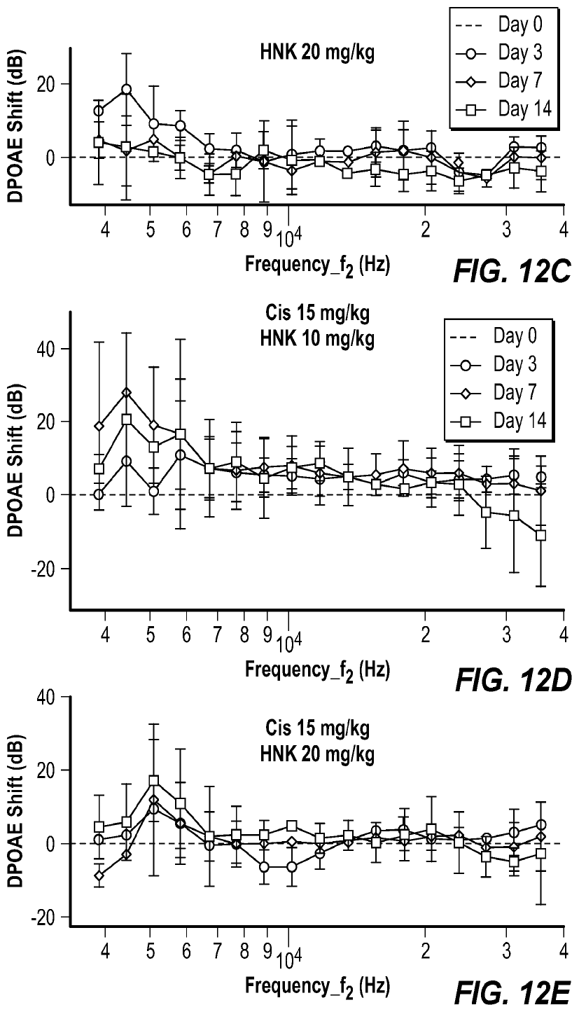

Distortion product oto-acoustic emissions (DPOAEs), which are an indication of outer hair cell (OHC) function, were measured in a separate series of experiments to estimate OHC protection by honokiol (HNK). (See FIG. 12). Cisplatin reduced the DPOAE amplitude at high frequencies as illustrated in FIG. 12A and in the grouped data of FIG. 12B, which is an indication of OHC damage. An apparent DPOAE amplitude drop was observed at frequencies over 16 kHz and more prominently at higher frequencies. When mice were pre-treated with HNK, DPOAE amplitude changes were less pronounced. (See FIGS. 12D and 12E). After pre-treatment with HNK at a dosage of 10 mg/kg, the DPOAE shift was observed only at frequencies of 27 kHz and higher with a much smaller amplitude. (See FIG. 12D). No DPOAE shift was observed when HNK as administered at a dosage of 20 mg/kg. (See FIGS. 12C and 12E), suggesting that HNK protected against cisplatin-induced OHC damage.

Immunofluorescent Histochemistry (IFHC) and Confocal Imaging Confirmed Honokiol Protection of Outer Hair Cells (OHCs) Against Cisplatin-Ototoxicity

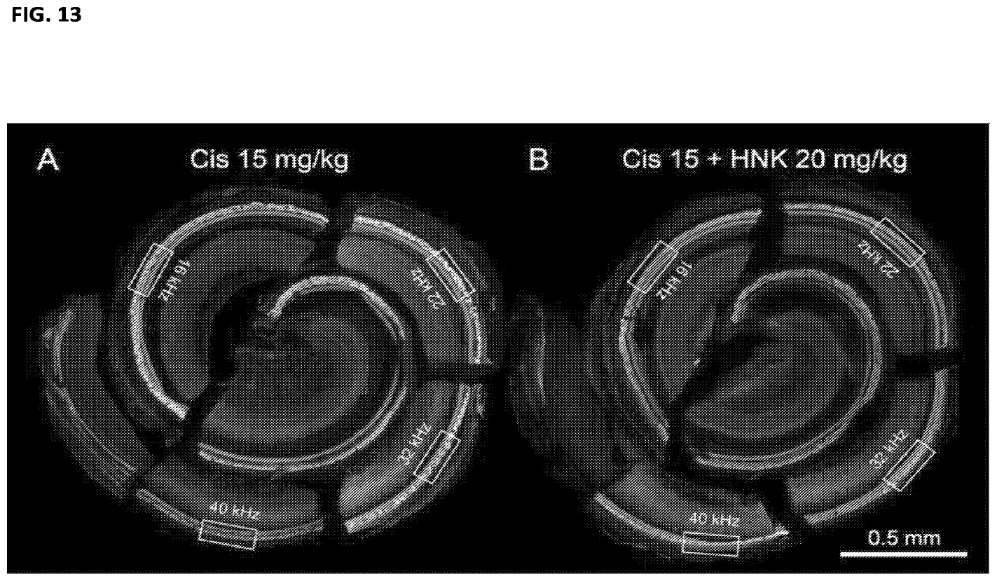

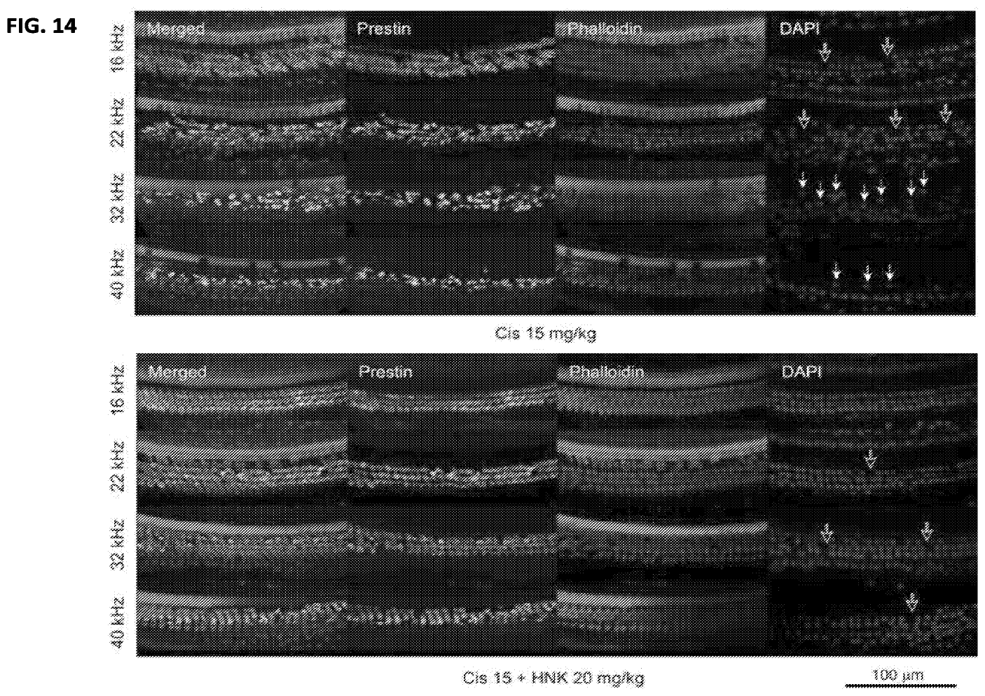

Immunofluorescent histochemistry (IFHC) of a cochlear whole mount was performed after the physiological studies described above using prestin antibody (for outer hair cells (OHC)), phalloidin (for hair bundle) and 4′,6-diamidino-2-phenylindole (DAPI) (for nuclei). FIG. 13 shows the staining of the entire cochleae and the selected locations with estimated frequency for high-resolution imaging in FIG. 14. Significant disruption of OHC organization and HC loss was observed at the basal ⅓ of the cochlea (estimated to be over 22 kHz) in the cisplatin-treated cochlea. (See FIG. 14A). HC loss can be indicated by the scarcity of remaining nuclei (white arrows). On the contrary, nearly intact OHC structure was maintained up to the 40 kHz region in HNK pre-treated cochlea (see FIG. 14B), with only sporadic loss of HCs (hollow arrows).

Weight Loss and Animal Survival: Honokiol Showed Obvious Health Protection Against Cisplatin Treatment in Terms of Reduced Weight Loss and Higher Survival Rate

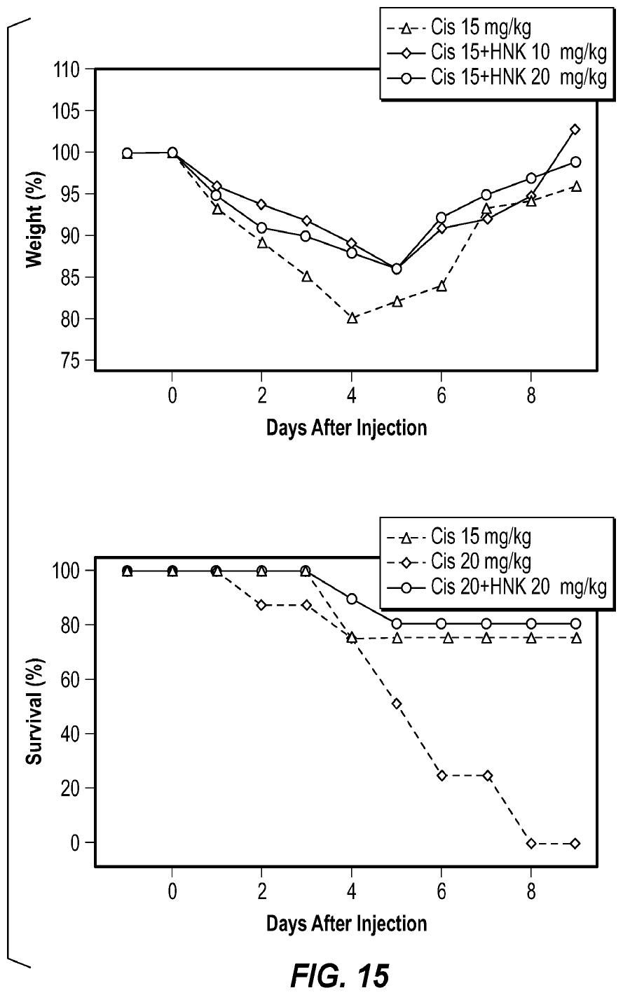

Serious health issues were observed in mice treated with cisplatin. (See FIG. 15). All the animals showed significant weight loss, and some animals were lost or removed from the study due to weight loss over 25%. With honokiol pre-treatment, however, the weight and animal loss were both significantly reduced. (See FIG. 15). The weight loss was slower and the maximum weight loss was about 15% instead of 20% when honokiol was administered. (See FIG. 15A). In the group of administered cisplatin at a dose of 20 mg/kg, all the animals were lost after 8 days while only 20% of the animals were lost when honokiol also was administered. (See FIG. 15B).

Summary: Cisplatin-Induced Outer Hair Cell (OHC) Loss which was Reversed by Honokiol Co-Application

In summary, HNK protected HEI-OC1 but not cancer cells from cisplatin-induced cell loss. When HNK was co-applied with cisplatin, expression levels of SIRT3 were increased and expression levels of cisplatin-induced apoptosis-related genes were decreased. Cisplatin induced a dose-dependent ABR threshold shift (TS), which diminished with HNK pre-treatment. HNK prevents cisplatin-induced DPOAE amplitude decrease, suggesting OHC protection. IFHC and confocal imaging confirmed HNK protection of OHCs against cisplatin-ototoxicity. HNK reduced weight loss and morbidity associated with cisplatin administration to mice.

Future aims include, demonstrating in tumor-bearing mice that HNK does not interfere with the tumor-suppressive effect of cisplatin, and demonstrating the benefit of the co-application of HNK in cisplatin chemotherapy for veterinary medicine.

### REFERENCES

1 Ohlemiller, K. K., Wright, J. S. & Dugan, L. L. Early elevation of cochlear reactive oxygen species following noise exposure. Audiol Neurootol 4, 229-236, doi:13846 (1999).
2 Huth, M. E., Ricci, A. J. & Cheng, A. G. Mechanisms of aminoglycoside ototoxicity and targets of hair cell protection. Int J Otolaryngol 2011, 937861, doi:10.1155/2011/937861 (2011).
3 Someya, S. & Prolla, T. A. Mitochondrial oxidative damage and apoptosis in age-related hearing loss. Mech Ageing Dev 131, 480-486, doi:10.1016/j.mad.2010.04.006 (2010).
4 Schacht, J., Talaska, A. E. & Rybak, L. P. Cisplatin and aminoglycoside antibiotics: hearing loss and its prevention. Anat Rec (Hoboken) 295, 1837-1850, doi:10.1002/ar.22578 (2012).
5 Kamogashira, T., Fujimoto, C. & Yamasoba, T. Reactive oxygen species, apoptosis, and mitochondrial dysfunction in hearing loss. Biomed Res Int 2015, 617207, doi:10.1155/2015/617207 (2015).
6 Wong, A. C. & Ryan, A. F. Mechanisms of sensorineural cell damage, death and survival in the cochlea. Front Aging Neurosci 7, 58, doi:10.3389/fnagi.2015.00058 (2015).
7 Howell, S. B. & Taetle, R. Effect of sodium thiosulfate on cis-dichlorodiammineplatinum(II) toxicity and antitumor activity in L1210 leukemia. Cancer Treat Rep 64, 611-616 (1980).
8 Rybak, L. P. & Kelly, T. Ototoxicity: bioprotective mechanisms. Curr Opin Otolaryngol Head Neck Surg 11, 328-333 (2003).
9 Treskes, M., Nijtmans, L. G., Fichtinger-Schepman, A. M. & van der Vijgh, W. J. Effects of the modulating agent WR2721 and its main metabolites on the formation and stability of cisplatin-DNA adducts in vitro in comparison to the effects of thiosulphate and diethyldithiocarbamate. Biochem Pharmacol 43, 1013-1019 (1992).
10 Dickey, D. T., Wu, Y. J., Muldoon, L. L. & Neuwelt, E. A. Protection against cisplatin-induced toxicities by N-acetylcysteine and sodium thiosulfate as assessed at the molecular, cellular, and in vivo levels. J Pharmacol Exp Ther 314, 1052-1058, doi:10.1124/jpet.105.087601 (2005).
11 Church, M. W., Blakley, B. W., Burgio, D. L. & Gupta, A. K. WR-2721 (Amifostine) ameliorates cisplatin-induced hearing loss but causes neurotoxicity in hamsters: dose-dependent effects. J Assoc Res Otolaryngol 5, 227-237, doi:10.1007/s10162-004-4011-z (2004).
12 Wang, J. et al. Local application of sodium thiosulfate prevents cisplatin-induced hearing loss in the guinea pig. Neuropharmacology 45, 380-393 (2003).
13 Planting, A. S. et al. Randomized study of a short course of weekly cisplatin with or without amifostine in advanced head and neck cancer. EORTC Head and Neck Cooperative Group. Ann Oncol 10, 693-700 (1999).
14 Ekborn, A. et al. High-dose Cisplatin with amifostine: ototoxicity and pharmacokinetics. Laryngoscope 114, 1660-1667, doi:10.1097/00005537-200409000-00030 (2004).
15 Rybak, L. P., Mukherjea, D., Jajoo, S. & Ramkumar, V. Cisplatin ototoxicity and protection: clinical and experimental studies. Tohoku J Exp Med 219, 177-186(2009).
16 Fried, L. E. & Arbiser, J. L. Honokiol, a multifunctional antiangiogenic and antitumor agent. Antioxid Redox Signal 11, 1139-1148, doi:10.1089/ARS.2009.2440 (2009).
17 Pillai, V. B. et al. Honokiol blocks and reverses cardiac hypertrophy in mice by activating mitochondrial Sirt3. Nat Commun 6, 6656, doi:10.1038/ncomms7656 (2015).
18 Yu, W., Dittenhafer-Reed, K. E. & Denu, J. M. SIRT3 protein deacetylates isocitrate dehydrogenase 2 (IDH2) and regulates mitochondrial redox status. The Journal of biological chemistry 287, 14078-14086, doi:10.1074/jbc.M112.355206 (2012).
19 Someya, S. et al. Sirt3 mediates reduction of oxidative damage and prevention of age-related hearing loss under caloric restriction. Cell 143, 802-812, doi:10.1016/j.cell.2010.10.002 (2010).
20 Kalinec, G. M., Webster, P., Lim, D. J. & Kalinec, F. A cochlear cell line as an in vitro system for drug ototoxicity screening. Audiol Neurootol 8, 177-189, doi:71059 (2003).
21 So, H. S. et al. Protective effect of T-type calcium channel blocker flunarizine on cisplatin-induced death of auditory cells. Hear Res 204, 127-139, doi:10.1016/j.heares.2005.01.011 (2005).
22 Rybak, L. P. Mechanisms of cisplatin ototoxicity and progress in otoprotection. Curr Opin Otolaryngol Head Neck Surg 15, 364-369, doi:10.1097/MOO.0b013e3282eee452 (2007).
23 Rybak, L. P., Whitworth, C. A., Mukherjea, D. & Ramkumar, V. Mechanisms of cisplatin-induced ototoxicity and prevention. Hear Res 226, 157-167, doi:10.1016/j.heares.2006.09.015 (2007).
24 Walters, B. J. et al. Pseudo-immortalization of postnatal cochlear progenitor cells yields a scalable cell line capable of transcriptionally regulating mature hair cell genes. Sci Rep 5, 17792, doi:10.1038/srep17792 (2015).
25 More, S. S. et al. Role of the copper transporter, CTR1, in platinum-induced ototoxicity. J Neurosci 30, 9500-9509, doi:10.1523/JNEUROSCI.1544-10.2010 (2010).
26 Iwamoto, Y. et al. Inactivation of cis-diamminedichloroplatinum (II) in blood and protection of its toxicity by sodium thiosulfate in rabbits. Cancer Chemother Pharmacol 15, 228-232 (1985).
27 Lin, J. W. et al. Honokiol traverses the blood-brain barrier and induces apoptosis of neuroblastoma cells via an intrinsic bax-mitochondrion-cytochrome c-caspase protease pathway. Neuro Oncol 14, 302-314, doi:10.1093/neuonc/nor217 (2012).
28 Wang, X. et al. Honokiol crosses BBB and BCSFB, and inhibits brain tumor growth in rat 9L intracerebral gliosarcoma model and human U251 xenograft glioma model. PLoS One 6, e18490, doi:10.1371/journal.pone.0018490 (2011).
29 Benedetti Panici, P. et al. Efficacy and toxicity of very high-dose cisplatin in advanced ovarian carcinoma: 4-year survival analysis and neurological follow-up. Int J Gynecol Cancer 3, 44-53 (1993).
30 Wu, Y. J., Muldoon, L. L. & Neuwelt, E. A. The chemoprotective agent N-acetylcysteine blocks cisplatin-induced apoptosis through caspase signaling pathway. J Pharmacol Exp Ther 312, 424-431, doi:10.1124/jpet.104.075119 (2005).
31 Videhult, P., Laurell, G., Wallin, I. & Ehrsson, H. Kinetics of Cisplatin and its monohydrated complex with sulfur-containing compounds designed for local otoprotective administration. Exp Biol Med (Maywood) 231, 1638-1645 (2006).
32 Riga, M. G. et al. Transtympanic injections of N-acetylcysteine for the prevention of cisplatin-induced ototoxicity: a feasible method with promising efficacy. Am J Clin Oncol 36, 1-6, doi:10.1097/COC.0b013e31822e006d (2013).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating and/or preventing hearing loss in a subject in need thereof, the method comprising administering to the subject an effective amount of a polyphenol therapeutic agent.

2. The method of claim 1, wherein the polyphenol therapeutic agent is administered via transtympanic injection, intracochlear injection, or an intra-cochlear drug deliver device.

3. The method of claim 1, wherein the hearing loss is an induced hearing loss.

4. The method of claim 1, wherein the hearing loss is noise-induced hearing loss.

5. The method of claim 1, wherein the hearing loss is drug-induced hearing loss due to ototoxicity.

6. The method of claim 5, wherein the ototoxicity is induced by an administered platinum-containing therapeutic.

7. The method of claim 6, wherein the platinum-based therapeutic is cisplatin, carboplatin, or oxaliplatin.

8. The method of claim 6, wherein the ototoxicity is induced by an administered antibiotic.

9. The method of claim 8, wherein the antibiotic is an aminoglycoside.

10. The method of claim 1, wherein the hearing loss is age-related hearing loss (ARHL).

11. The method of claim 1, wherein the polyphenol therapeutic agent is a bi-phenol agent.

12. The method of claim 11, wherein the bi-phenol agent is honokiol.

13. The method of claim 1, wherein the subject in need thereof has cancer.

14. The method of claim 13, wherein the cancer is selected from cervical cancer, prostate cancer, and colon cancer.

15. The method of claim 13, further comprising administering to the subject a platinum-containing chemotherapeutic agent.
